(19) United States
(12) Patent Application Publication
(10) Pub. No.: US 2024/0358232 A1
(43) Pub. Date: Oct. 31, 2024

(54) SYSTEMS AND METHODS FOR SELECTIVELY RIGIDIZING A FLEXIBLE INSTRUMENT

(71) Applicant: INTUITIVE SURGICAL OPERATIONS, INC., Sunnyvale, CA (US)

(72) Inventors: Miles S. Joseph, Sunnyvale, CA (US); Bruce M. Schena, Sunnyvale, CA (US)

(21) Appl. No.: 18/647,595

(22) Filed: Apr. 26, 2024

Related U.S. Application Data
(60) Provisional application No. 63/498,766, filed on Apr. 27, 2023.

(51) Int. Cl. A61B 1/00; A61B 1/005

(52) U.S. Cl. CPC A61B 1/00098; A61B 1/009

(57) ABSTRACT

A system may comprise a flexible delivery device including a tool channel extending therethrough and an elongated instrument configured to extend within the tool channel. The elongated instrument may include a flexible section and a rigidizable section. The system may also include a selective rigidization system at least partially extending within the elongated instrument and a sensor system configured to determine position information for the rigidizable section relative to a distal portion of the delivery device. The selective rigidization system, responsive to the position information, may be configured to transition a portion of the rigidizable section of the instrument from a bendable state to a rigid state.

### CROSS-REFERENCED APPLICATIONS

This application claims priority to and benefit of U.S. Provisional Applications No. 63/498,766 filed Apr. 27, 2023 and entitled “Systems and Methods for Selectively Rigidizing a Flexible Instrument,” which is incorporated by reference herein in its entirety.

### FIELD

Examples described herein relate to systems and methods for selectively rigidizing a flexible instrument. More particularly, example systems and methods may selectively rigidize a portion of a flexible instrument that extends distally of a flexible catheter.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. Some minimally invasive medical instruments may be used to perform tasks that require applying force to external structures within the patient anatomy. Systems and methods are needed that allow a minimally invasive instrument to be flexible enough to navigate a tortuous anatomic path but rigid enough to apply forces within the anatomy at a target location.

### SUMMARY

The following presents a simplified summary of various examples described herein and is not intended to identify key or critical elements or to delineate the scope of the claims.

In some examples, a system may comprise a flexible delivery device including a tool channel extending therethrough and an elongated instrument configured to extend within the tool channel. The elongated instrument may include a flexible section and a rigidizable section. The system may also include a selective rigidization system at least partially extending within the elongated instrument and a sensor system configured to determine position information for the rigidizable section relative to a distal portion of the delivery device. The selective rigidization system, responsive to the position information, may be configured to transition a portion of the rigidizable section of the instrument from a bendable state to a rigid state.

In some examples, a system may comprise a control system, a flexible delivery device including a tool channel extending therethrough, and an elongated flexible instrument configured to extend within the tool channel. The elongated instrument may include a rigidizable section. The system may also include a sensor system configured to determine position information for the rigidizable section of the instrument relative to a distal portion of the delivery device. The control system may comprise programmed instructions to automatically transition the rigidizable section from a bendable state to a rigid state responsive to the position information.

In some examples, a system may comprise a flexible delivery device including a tool channel extending therethrough, a flexible elongated instrument configured to extend within the tool channel, and a selective rigidization system including a clamping member within the tool channel. The clamping member may be configured to selectively apply a clamping force to the elongated instrument within the tool channel. The system may also include a sensor system configured to determine position information of the flexible elongated instrument relative to a distal portion of the delivery device. The selective rigidization system, responsive to the position information, may be configured to apply the clamping force to the elongated instrument to immobilize a portion of the flexible elongated instrument in contact with the clamping member.

In some examples, a system may comprise a flexible delivery device including a tool channel extending therethrough and an elongated instrument configured to extend within the tool channel. The elongated instrument may include a flexible section and a rigidizable section. The rigidizable section may house a magnetorheological fluid. The system may also comprise a selective rigidization system including a magnet system at a distal portion of the delivery device. The magnetorheological fluid may be responsive to the magnet system to transition a portion of the rigidizable section from a bendable state to a rigid state as the elongated instrument is extended distally relative to the magnet system.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a patient anatomy with a medical instrument system, according to some examples.

FIG. 2 is a side view of a distal end of a medical instrument system, according to some examples.

FIG. 3 is a side view of a distal end of a medical instrument system with a shape sensor, according to some examples.

FIG. 4 is a side view of a distal end of a medical instrument system with a position sensor, according to some examples.

FIG. 5 is a side view of a distal end of an instrument, according to some examples.

FIG. 6 is a side view of a distal end of an instrument, according to some examples.

FIG. 7 is a side view of a distal end of a medical instrument system, according to some examples.

FIG. 8 is a side view of a distal end of a medical instrument system, according to some examples.

FIG. 9 is a flowchart illustrating a method for selectively rigidizing a flexible instrument.

FIG. 10 illustrates a distal of a medical device with rigidizable elements, according to some examples.

FIGS. 11A and 11B illustrate rigidizable elements, according to some examples.

FIG. 12 is a robotically-assisted medical system, according to some examples.

FIG. 13A is a simplified diagram of a medical instrument system according to some embodiments.

FIG. 13B is a simplified diagram of a medical instrument with an extended medical instrument according to some embodiments.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

In various examples provided in this disclosure, medical instrument systems may include a flexible delivery device, such as a catheter, through which a flexible instrument may be extended. Various systems and methods are described that allow the flexible instrument to bend and deflect as the flexible instrument navigates tortuous anatomic passageways to an anatomic intervention site but to rigidize portions of the flexible instrument and/or the flexible delivery device to resist buckling and lateral deflections when applying a force at the interventional site. Although the examples provided herein may be used for procedures within the gastrointestinal system, it is understood that the described technology may be used in performing procedures in artificially created lumens or any endoluminal passageway or cavity, including in a patient trachea, colon, intestines, stomach, liver, kidneys and kidney calices, brain, heart, lungs, circulatory system including vasculature, fistulas, and/or the like.

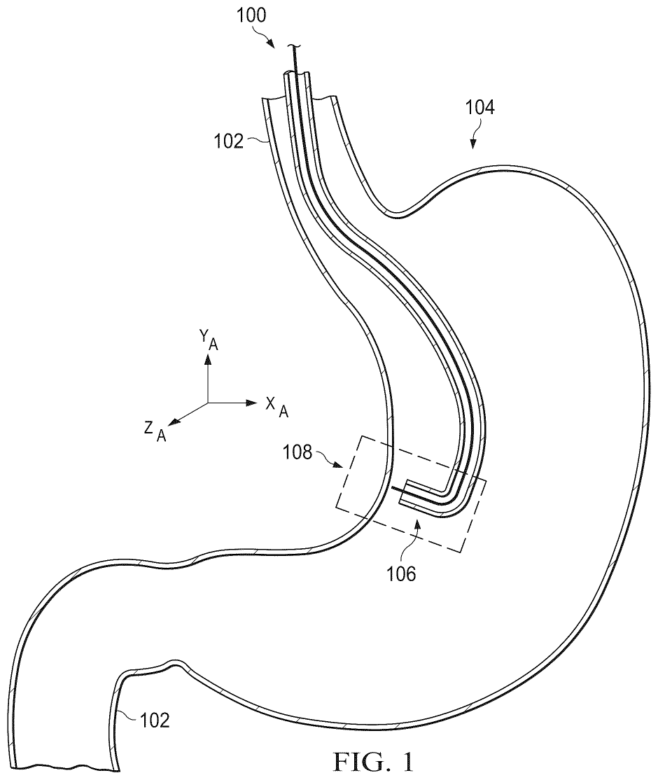

FIG. 1 illustrates a medical instrument system 100 extending within anatomic passageways 102 of an anatomical structure 104. In some examples the anatomic structure 104 may be a stomach. The anatomic structure 104 has an anatomical frame of reference (X_{A}, Y_{A}, Z_{A}). A distal end portion 106 of the medical instrument system 100 may be advanced into an anatomic opening (e.g., a patient mouth) and through tortuous anatomic passageways, including the anatomic passageway 102, to perform a medical procedure at or near target tissue located in a region 108 of the anatomic structure 104 using any of the methods or systems described herein.

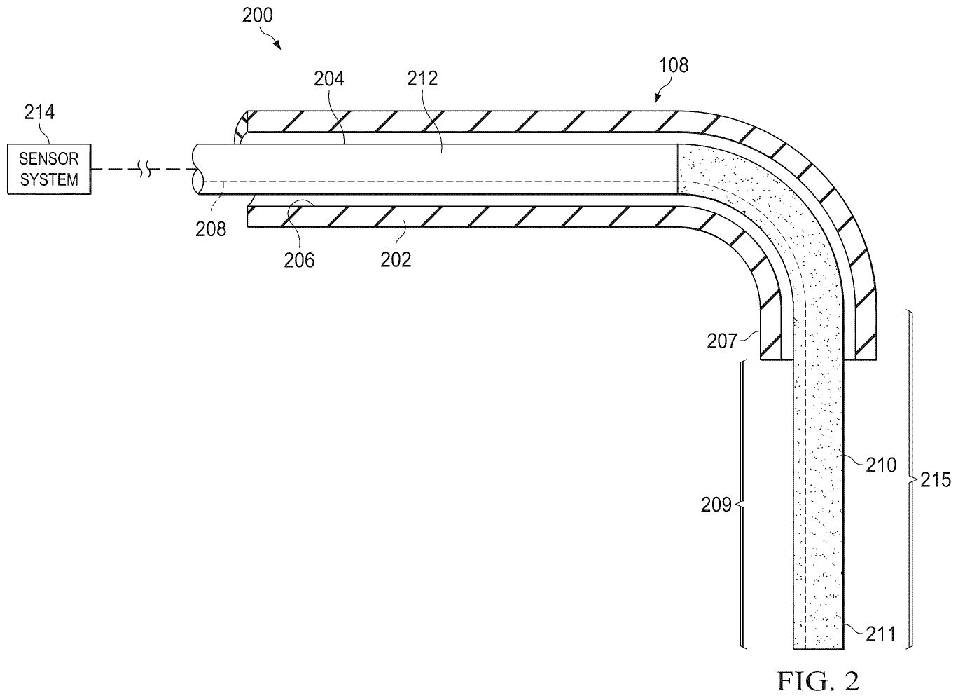

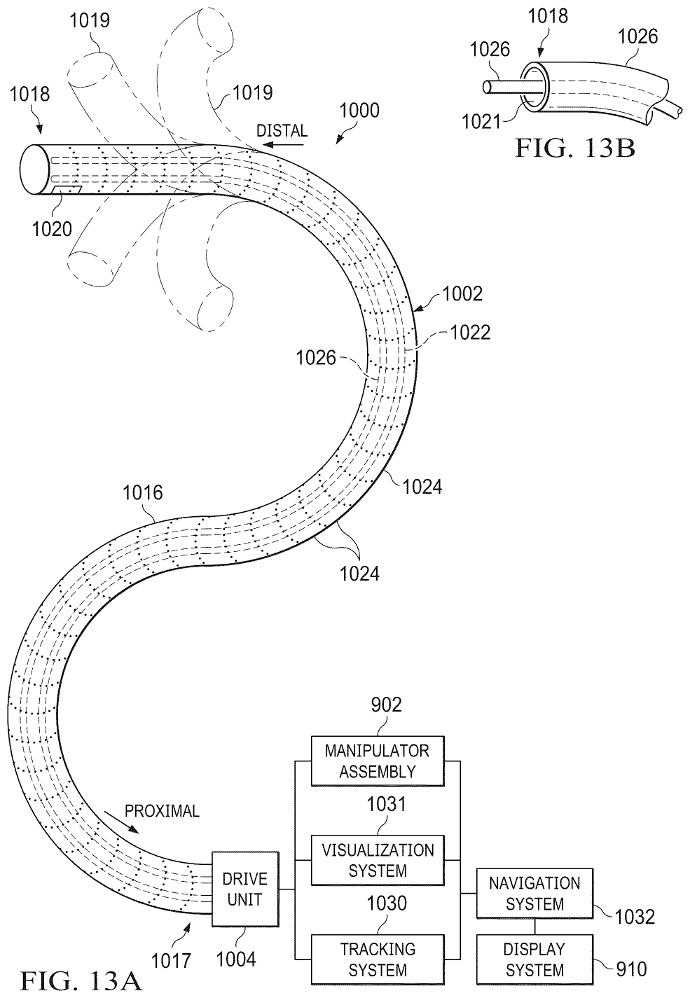

FIG. 2 provides a schematic view of a distal portion of a medical instrument system 200 (e.g., the medical instrument system 100) which may include a flexible delivery device 202 (e.g., the elongate device 1002 of FIG. 13A) and an instrument 204 extending within a channel 206 of the flexible delivery device 202. The flexible delivery device 202 may have a distal end portion 207 at which a distal end of the channel 206 has an opening, allowing an extension portion 209 of the instrument 204 to extend distally of the distal end portion 207 of the flexible delivery device 202. In alternative examples, a delivery device may be rigid. In some embodiments, the flexible delivery device 202 may have a plurality of channels 206 allowing passage for respective instruments 204.

The instrument 204 may include a distal end portion 211, and an extension portion 209 of the instrument 204 may include the distal end portion 211 when the instrument 204 is extended from the channel 206. For example, the instrument 204 may be advanced through the channel 206 and the extension portion 209 of the instrument 204 may be advanced distally of the distal end portion 207 of the delivery device 202 and into contact with tissue. The instrument 204 may include any of various tools, instruments, or end effectors. For example, the instrument 204 may include a biopsy or tissue sampling tool (e.g., needle or forceps), a suturing tool, an ablation tool, an imaging tool, grasping instrument, cutting instrument, gripping instrument, a medication delivery device, and/or another type of surgical, diagnostic, or therapeutic device. The instrument 204 may include a selective rigidization system 208 and a rigidizable section 210 that may be dynamically transitioned from a flexible or bendable state to a rigid state based on the selective rigidization system 208. The instrument 204 may also include a flexible section 212 proximal of the rigidizable section 210 that may remain in a flexible state when the rigidizable section 210 is in the rigid state. The flexible section 212 may allow the instrument 204 to move in an axial direction (e.g., in/out) relative to the delivery device 202 and/or in a rotational direction relative to the delivery device 202 while the rigidizable section 210 is in a rigid state. Maintaining the flexibility of the flexible section 212 may also allow the flexible delivery device 202 to be moved, articulated, or otherwise change position or pose, while the rigidizable section 210 of the instrument 204 is in the rigid state.

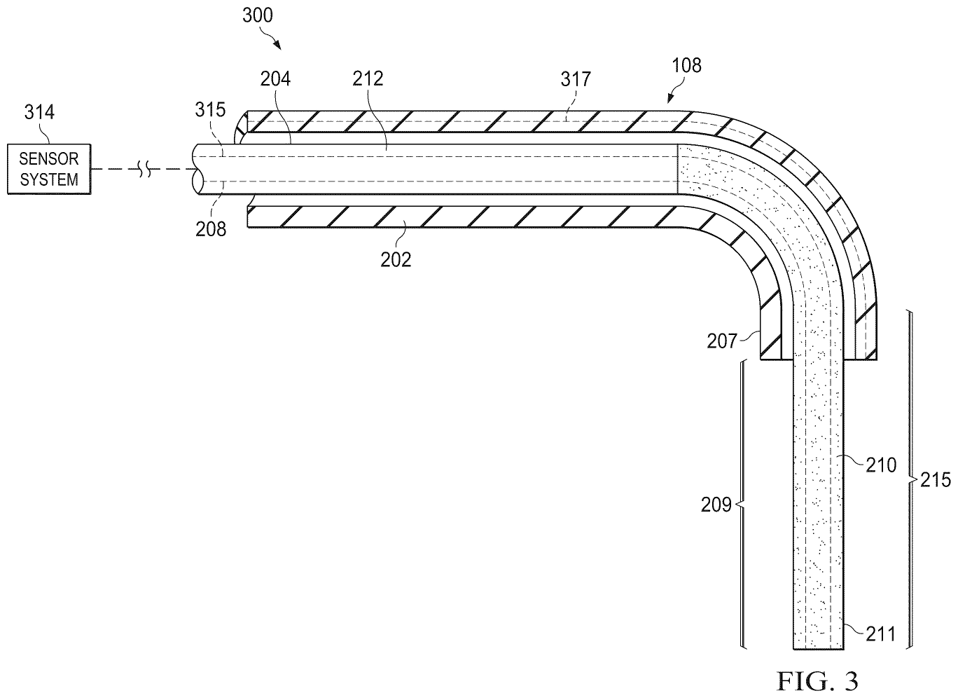

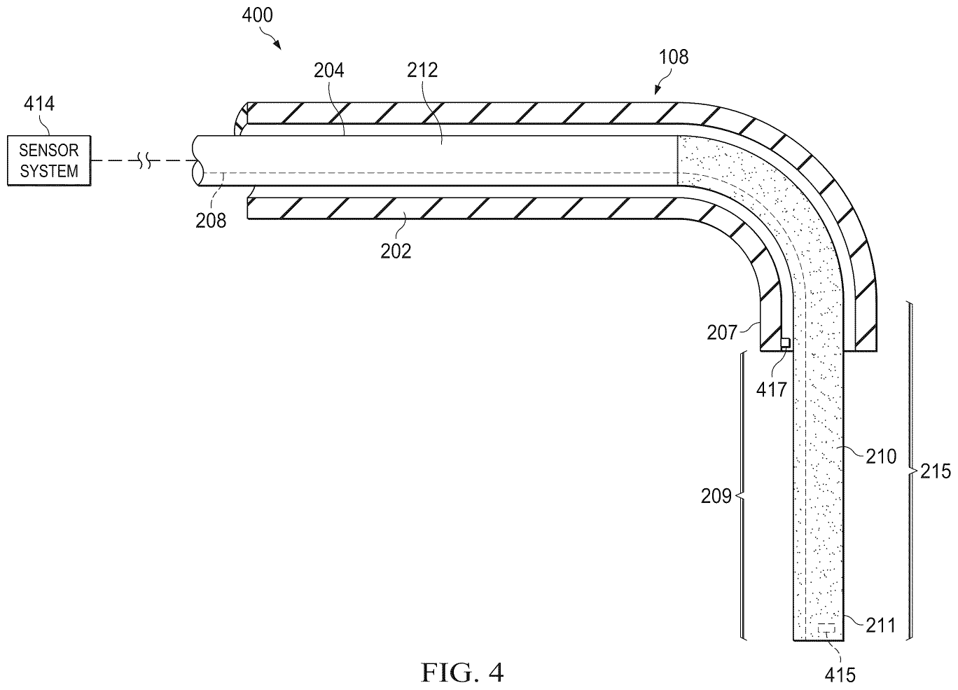

The medical instrument system 200 may also include a sensor system 214 that provides sensor data indicating position, shape, pose, or other information about the instrument 204 and/or the delivery device 202. The sensor data from the sensor system 214 may indicate, for example, whether the instrument 204 has extended from the channel 206 to an area distal of the distal end portion 207, the length of the extension portion 209 of the instrument 204, and/or the shape of the extension portion 209 of the instrument 204. The sensor system may include, for example, a shape sensor such as an optical fiber shape sensor as described with respect to FIG. 3 or an electromagnetic position sensor as described with respect to FIG. 4. In other examples, the sensor system may additionally or alternatively include an encoder on a motor driving extension/retraction motion of the instrument 204. In other examples, the sensor system may additionally or alternatively include an imaging sensor, such as a camera and markers on the extension portion 209 of the instrument 204 visible by the imaging sensor.

The selective rigidization system 208 may include various components to dynamically cause a selected portion 215 of the rigidizable section 210 of the instrument 204 to change from a bendable state to a rigid state. For example, the selective rigidization system 208 may include a rigidizable element, a control system, and trigger mechanism. The selected portion 215 of the rigidizable section 210 may be anywhere along the length of the rigidizable section 210. For example, the selected portion 215 for rigidization may be the entire length of the extension portion 209 that extends distally of the delivery device 202. In other examples, the selected portion 215 for rigidization may be a partial length of the extension portion 209. In some examples, the selected portion 215 for rigidization may include a portion of the rigidizable section 210 proximal of the extension portion 209 that remains within the delivery device 202 (e.g., the selected portion 215 for rigidization may include a region positioned proximal to a distal end face of the delivery device 202). In some examples, the selected portion 215 for rigidization may include an extension portion 209 extending distally of the delivery device 202 and a portion of the rigidizable section 210 extending within a distal end face of the delivery device 202. In some examples, the selected portion 215 for rigidization may include predetermined segments, that may be continuous or discontinuous, along the rigidizable section 210. In some examples, the selected portion 215 for rigidization may include a midportion of the rigidizable section 210, between the distal end portion 211 of the instrument 204 and the flexible section 212 of the instrument 204. In some examples, the extension portion 209 may be rigidized in a generally straight configuration, but in other examples, the extension portion 209 may be rigidized in a curved shape or other non-linear configuration.

The selective rigidization system 208 may include a control system (which may be a part of a robotically-assisted control system such as control system 912) which comprises programmed instructions for transitioning the rigidizable section 210 from the bendable state to the rigid state in response to a trigger mechanism and/or for transitioning the rigidizable section 210 from the rigid state to the bendable state. The trigger mechanism may be triggered based on information from the sensor system 214 (e.g., information relating to position and/or orientation of portions of the instrument 204 relative to the delivery device 202), movement of the rigidizable section 210, and/or based on operator control. The selective rigidization system 208 may also include a rigidizable element located in or on the instrument 204 and/or on the delivery device 202. As described in examples below, the rigidizable element may include, for example, mechanical, pneumatic, hydraulic, magnetic, or other types of rigidizing mechanisms.

FIG. 3 provides a schematic view of a distal portion of a medical instrument system 300 (e.g., the medical instrument system 100) which may be similar to medical instrument system 200, with differences as described. In this example, the medical instrument system 300 includes a sensor system 314 (which may be an example of sensor system 214), including a shape sensor 315 extending to the distal end portion 211 of the instrument 204 and a shape sensor 317 extending to the distal end portion 207 of the flexible delivery device 202. Shape sensor 315, 317 may optionally be optical fiber shape sensors (e.g., one or more optical fibers or fiber bundles having fiber Bragg gratings). In some examples, the optical fiber may have a diameter of approximately 200 μm. In other examples, the dimensions may be larger or smaller. The optical fiber of shape sensor 315 may form a fiber optic bend sensor for determining the shape of flexible instrument 204 or optionally, a portion of flexible instrument 204 such as the rigidizable section 210. The measured shape from the shape sensor 315 may also provide the position and orientation of the distal end portion 211 of the instrument 204. The optical fiber of shape sensor 317 may form a fiber optic bend sensor for determining the shape of flexible delivery device 202 and, consequently, the position and orientation of the distal end portion 207 of the flexible delivery device 202. The length and orientation of the extension portion 209 of the instrument 204 relative to the flexible delivery device 202 may be determined by comparing the shape data from the shape sensors 315 and 317. For, example, when the distal end portion 211 of the instrument 204 is extended from the delivery device 202, the shape data from sensor 315 relative to the shape data 317 will indicate the distance that the position of the distal end portion 211 of the instrument 204 is displaced from the position of the distal end portion 207 of the delivery device 202 as well as their relative orientations, and thereby provide position and orientation information of the extension portion 209. In one alternative, optical fibers including Fiber Bragg Gratings (FBGs) may be used to provide strain measurements in structures in one or more dimensions. Sensors in some examples may employ other suitable strain sensing techniques, such as Rayleigh scattering, Raman scattering, Brillouin scattering, and Fluorescence scattering.

FIG. 4 provides a schematic view of a distal portion of a medical instrument system 400 (e.g., the medical instrument system 100) which may be similar to medical instrument system 200, with differences as described. In this example, the medical instrument system 400 includes a sensor system 414 (which may be an example of the sensor system 214), including a position sensor 415 on the instrument 204 and a position sensor 417 on the flexible delivery device 202.

In some examples, the position sensors 415, 417 may be electromagnetic (EM) sensors that may include one or more conductive coils and/or magnets that may be subjected to an externally generated electromagnetic field. Each coil and/or magnet of the EM sensor system then produces an induced electrical signal having characteristics that depend on the position and orientation of the coil and/or magnet relative to the externally generated electromagnetic field. In some examples, position sensor system 414 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. In some examples, the position sensor 415 may be positioned at the distal end portion 211 of the instrument 204, and the position sensor 417 may be positioned at the distal end portion 207 of the flexible delivery device 202. The length and orientation of the extension portion 209 of the instrument 204 may be determined by comparing the position data from the position sensors 415 and 417. For, example, when the distal end portion 211 of the instrument 204 is extended from the delivery device 202, the position data from sensor 415 relative to the sensor 417 will indicate the distance that the position of the distal end portion 211 of the instrument 204 is displaced from the position of the distal end portion 207 of the delivery device 202 as well as their relative orientations, and thereby provide position and orientation information of the extension portion 209.

In some examples, the position sensors 415, 417 may communicate with each other to determine the displacement of the sensors. For example, one of the position sensors may be an EM coil sensor that communicates with and the other sensor which may include a magnet. In some examples, only one position sensor may be used. As one example, the position sensor 415 may be omitted and the position sensor 417 may include an optical sensor that senses when the distal end portion 211 has passed a known location. As another example, the position sensor 417 may be omitted and the position sensor 415 may be an EM sensor whose position may be determined relative to a known location of the delivery device 202. In some examples, the position sensors 415, 417 may be located near the distal tips of the respective components. In other examples, the position sensors may be located more proximally or sense a position that is proximal to the distal tip. For example, an optical sensor located on the delivery device 202 at a known fixed proximal location may sense a marker that is known fixed proximal location on the instrument 204.

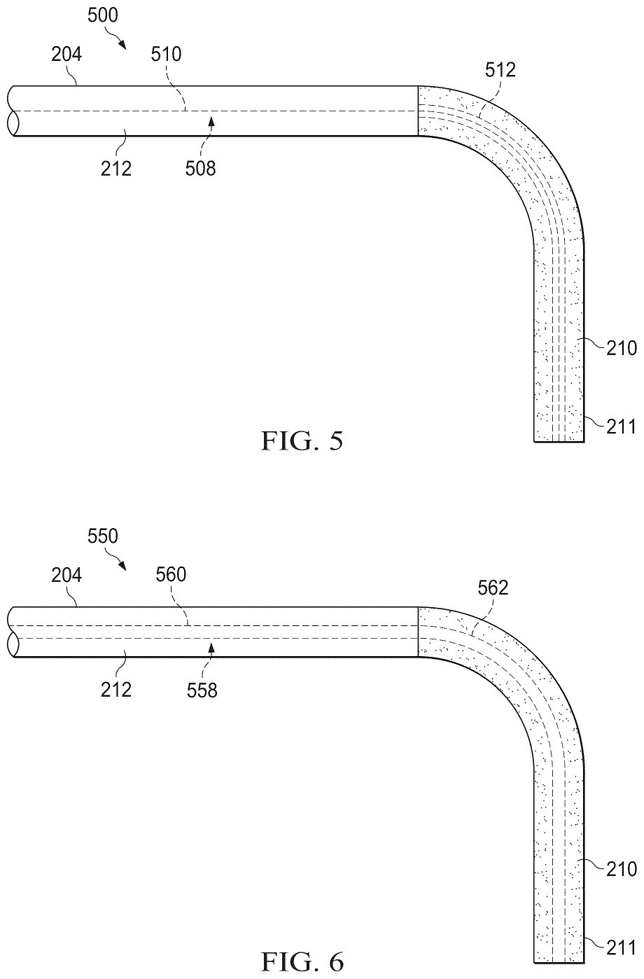

FIG. 5 provides a schematic view of a distal portion of an instrument 500 which may be similar to instrument 204, with differences as described. In this example, the instrument 500 includes selective rigidization system 508 (e.g., the selective rigidization system 208) including an elongated control member 510 such as a wire, tendon, or cable extending through the flexible section 212 of the instrument 500 and into the rigidizable section 210 of the instrument 500. The elongated control member 510 may terminate, for example, at or near the distal end portion 211 of the instrument 500. The selective rigidization system 508 may also include a rigidizable element 512 which may include, for example, a spring, a coil pipe, or a series of compressible linkages through which the control member 510 extends. A distal end of the rigidizable element 512 may be fixed at or near the distal end portion 211, and a proximal end of the rigidizable element 512 may be fixed at or near the proximal end of the rigidizable section 210. To transition the rigidizable section 210 from a bendable state to a rigid state, the control member 510 may be tensioned to compress the rigidizable element 512 into a rigid configuration. In other examples, a control member such as a cable may be used to expand a coil pipe to transition the rigidizable element to a rigid configuration. With the rigidizable section 210 held in the rigid state, the flexible section 212 and the control member 510 may remain in a bendable state. In the rigid state, the rigidizable section 210 may be used to apply forces at target locations and resist buckling or deflection, while the flexible section 212 extending within the delivery device 202 remains flexible to avoid causing unwanted motion or forces on the delivery device 202. The flexibility of the flexible section 212 in the rigid state also allows the instrument 500 to be translated and/or rotated relative to the delivery device 202 while the rigidizable section 210 remains in the rigid configuration to be able to apply forces at target locations. To transition the rigidizable section 210 from the rigid state to the bendable state, tension on the control member 510 may be relieved, and the rigidizable element 512 may be uncompressed. In various alternatives, the selective rigidization system may include a plurality of control members and a plurality of rigidizable elements to provide localized, independent, rigidization control within the rigidizable section.

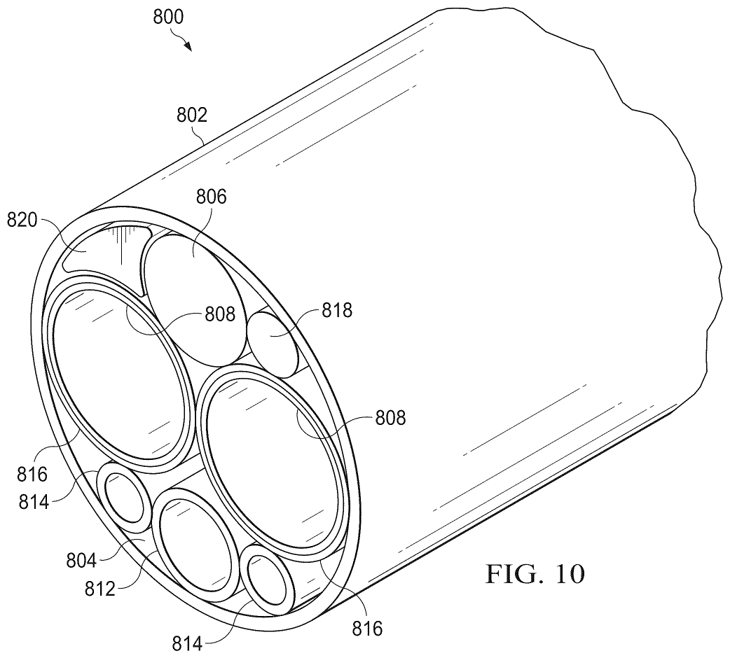

FIG. 6 provides a schematic view of a distal portion of an instrument 550 which may be similar to instrument 204, with differences as described. In this example, the instrument 550 includes a selective rigidization system 558 (e.g., the selective rigidization system 208) including a tubular member 560 extending through the flexible section 212 to a proximal end of the rigidizable section 210. The selective rigidization system 558 may also include a rigidizable element 562 which may include, for example, an expandable or inflatable chamber or series of chambers. The inflatable chamber may extend, for example, from the distal end portion 211 to a proximal end of the rigidizable section 210. To transition the rigidizable section 210 from a bendable state to a rigid state, a fluid such as air, water, or saline may be delivered through the tubular member 560 to the rigidizable element 562 to inflate or expand the chamber into a rigid configuration. Once inflated or expanded, the chamber of the rigidizable element 562 may be isolated from the tubular member 560 (e.g., with a seal or valve) so that the rigidizable section 210 remains in a rigid state. While the rigidizable section 210 is in the rigid state, the tubular member 560 and the flexible section 212 may remain in a flexible state. In the rigid state, the rigidizable section 210 may be used to apply forces at target locations and resist buckling or deflection while the flexible section 212 extending within the delivery device 202 remains flexible to avoid causing unwanted motion or forces on the delivery device. The flexibility of the flexible section 212 in the rigid state also allows the instrument 550 to be translated and/or rotated relative to the delivery device 202 while the rigidizable section 210 remains in the rigid configuration to be able to apply forces at target locations. To transition the rigidizable section 210 from the rigid state to the bendable state, the fluid may be evacuated from the chamber of the rigidizable element. In various alternatives, the selective rigidization system may include a plurality of tubular members and chambers to provide localized, independent, rigidization control within the rigidizable section. In some examples, the selective rigidization system may include a pneumatic actuator to deliver and evacuate air from the tubular member or may include a hydraulic actuator to deliver a pressurized fluid and evacuate a fluid from the tubular member. In some examples, the rigidizable element 562 may function as a vacuum chamber to provide rigidization. Other examples of internal rigidization elements are described below for FIG. 10.

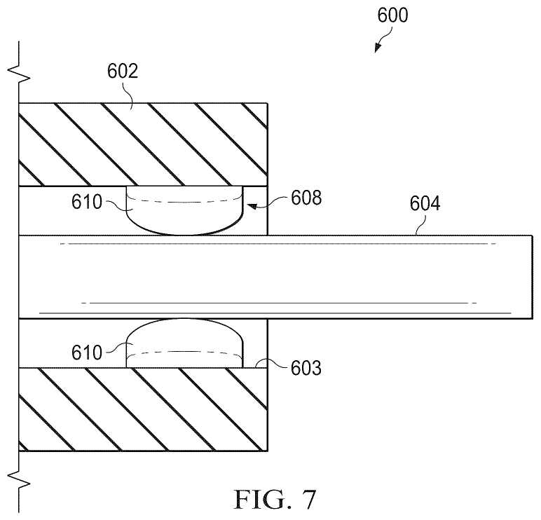

In some examples, some or all of the components of the selective rigidization system may be external to the instrument. FIG. 7 provides a schematic view of a distal portion of a medical instrument system 600 (e.g., the medical instrument system 100) which may be similar to medical instrument system 200, with differences as described. In this example, a delivery device 602 (e.g., the delivery device 202) includes a channel 603 through which an instrument 604 extends. The medical instrument system 600 may also include a selective rigidization system 608 (e.g., the selective rigidization system 208) including a rigidizing element 610 such as a clamping member that may be selectively expanded into contact with the instrument 604. In some examples, the clamping member 610 may be an inflatable sphincter that extends at least partially around the inner circumference of the channel 603. The lateral forces on the instrument 604 provided by the clamping member 610 may create a rigid state in the rigidizable section of the instrument 604 distal of the clamping member, including the extension portion that extends outside of the channel 603. In the rigid state, the extension portion of the instrument 604 may resist buckling and withstand lateral forces in the patient anatomy. In some examples, the selective rigidization system 608 may also include an internal rigidization element (e.g., rigidization elements within the instrument), as described in other examples, to provide further rigidization of the rigidizable section. To transition the rigidizable section of the instrument 604 from a bendable state to a rigid state, the inflatable sphincter may be filled with a fluid such as air, water, or saline delivered through a lumen in the delivery device or a tube extending through the channel 603. To transition the rigidizable section of the instrument from the rigid state to the bendable state, the clamping member 610 may be disengaged from the instrument 604. For example, the fluid may be evacuated from the inflatable sphincter of the clamping member 610. In various alternatives, the clamping member may include other types of selectively extendable and retractable components to clamp the instrument 604.

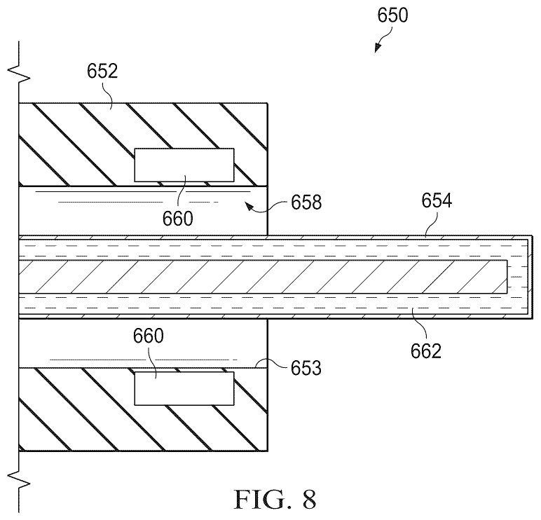

In some examples, components of the selective rigidization system may be both internal and external to the instrument. FIG. 8 provides a schematic view of a distal portion of a medical instrument system 650 (e.g., the medical instrument system 100) which may be similar to medical instrument system 200, with differences as described. In this example, a delivery device 652 (e.g., the delivery device 202) includes a channel 653 through which an instrument 654 extends. The medical instrument system 600 may also include a selective rigidization system 658 (e.g., the selective rigidization system 208) including rigidizing element 660 such as a magnetic member, and a magnetorheological fluid 662 that extends within a chamber or lumen of the rigidizable section of the instrument 654. In some examples, the magnetic member 660 may be a ring magnet or a series of discrete magnets arranged at least partially around the inner circumference of the channel 653. The magnetic member 660 may activate the magnetorheological fluid 662 as the fluid filled portion of the instrument 654 extends distally of the magnetic member. The activated magnetorheological fluid 662 may create a rigid state in the rigidizable section of the instrument 654 distal of the magnetic member 660, including the extension portion that extends outside of the channel 653. In the rigid state, the extension portion may resist buckling and withstand lateral forces in the patient anatomy. To transition the rigidizable section of the instrument from the rigid state to the bendable state, the magnet member 660 may be deactivated or the instrument portion that includes the magnetorheological fluid 662 may be retracted proximally of the magnetic member 660.

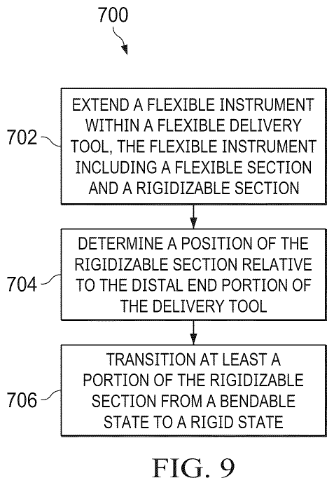

FIG. 9 is a flow chart illustrating a method 700 for selectively rigidizing a flexible instrument. The method 700 is illustrated as a set of operations or processes that may be performed in the same or in a different order than the order shown. One or more of the illustrated processes may be omitted in some examples of the method. Additionally, one or more processes that are not expressly illustrated in FIG. 7 may be included before, after, in between, or as part of the illustrated processes. In some examples, one or more of the processes of method 700 may be implemented, at least in part, by a control system executing code stored on non-transitory, tangible, machine-readable media that when run by one or more processors (e.g., the processors of a control system) may cause the one or more processors to perform one or more of the processes

At a process 702, a flexible instrument (e.g. instrument 204) may be extended within a flexible delivery device (e.g. delivery device 202). The flexible state of the flexible instrument and the flexible delivery device may allow them to navigate tortuous anatomic passageways. Even if the tortuous passageway is unchanging, as the instrument and delivery device are advanced, different sections of the flexible instrument and flexible delivery device may bend, unbend, and twist to conform to the passageway.

At a process 704, a position of a rigidizable section of the flexible instrument (e.g., rigidizable section 210) may be determined relative to a distal end portion of the delivery device. For example sensor information from the sensor system 214 may indicate that the distal end portion 211 of the instrument 204 has extended past the distal end portion 207 of the delivery device 202 and may further indicate the length and/or shape of the extension portion 209. The sensor information may be received, for example, from a shape sensor such as an optical fiber shape sensor as described in FIG. 2 or an electromagnetic position sensor as described in FIG. 3. In other examples, the sensor information may additionally or alternatively be received from an encoder on a motor driving extension/retraction motion of the instrument 204 or from an imaging sensor, such as a camera that observes markers or other known structures of the extension portion 209 of the instrument 204 visible by the imaging sensor.

At a process 706, at least a portion of the rigidizable section of the instrument may be transitioned from a bendable state to a rigid state. For example, a portion of the rigidizable section 210 of the instrument 204 may be transitioned from a bendable or flexible state to a rigid state by the selective rigidization system 208. In the rigid state, the rigidizable section 210 may be used to apply forces in the anatomic region (e.g. drive a suture needle, puncture a tissue wall) and may resist buckling or lateral deflections. With the rigidizable section 210 transitioned to the rigid state, the rigidizable section 210 may be considered isolated from the flexible section 212, which may remain flexible. The flexible section 212 may allow the instrument 204 to move in an axial direction (e.g., in/out) and/or in a rotational direction while the rigidizable section 210 is in a rigid state. Maintaining the flexibility of the flexible section 212 may also avoid causing unwanted motion or forces on the delivery device and allow the flexible delivery device 202 to be moved, articulated, or otherwise change position or pose, while the rigidizable section 210 of the instrument 204 is in the rigid state. In some examples, only the extension portion 209 (e.g., as determined by the sensor information) of the rigidizable section 210 may be selected to transition to a rigidized state. In some examples, the extension portion 209 as well as a predetermined length of the instrument 204 remaining within the delivery device 202 may be selected for rigidization. The transition may be triggered by the selective rigidization system's recognition that the distal end portion 211 of the instrument 204 has extended distally of the distal end portion 207 of the delivery device 202. In some examples, the transition may be triggered by the selective rigidization system's recognition that the instrument 204 has extended past the delivery device 202 by a predetermined distance. The predetermined distance may be based on the stiffness and/or the diameter of the instrument. For example the predetermined distance may be a length greater than a diameter of the instrument 204. As described in the various examples above, the selective rigidization system 208 may include, for example, mechanical (e.g., tensioned control members with a rigidizing element), pneumatic, hydraulic, magnetic, or other types of rigidizing systems to transition the rigidizable section to and from a rigid state. The triggering of the transition may be automatic, for example, if a control system of a robot-assisted medical system monitors the sensor information and recognizes the extension of the instrument based on the sensor information. As the instrument is extended, a manipulation system of the robot-assisted system may initiate the rigidization (e.g., tensioning a control member, activating a pneumatic/hydraulic system, or passively activating the magnetic rigidizing system). Likewise, automatic transition from the rigid state to a flexible state may be responsive to sensor information indicating the full (or predetermined amount of) retraction of the instrument 204 into the delivery device 202. In alternative embodiments, the selective rigidization may not be automated but may be actuated by an operator, based on the operator's expertise or based on displayed or otherwise presented sensor information. In some examples, the sensor information (e.g., the shape sensor) may provide an indication of excessive deflection or strain and send a recommendation to the user to activate rigidization. In some examples, the system may provide an indicator to the user of the distance of extension and may provide a recommendation to the user to activate rigidization

FIG. 10 illustrates a distal end of a medical device 800 which includes various rigidizable elements that may be used to rigidize the medical device 800. In some examples, the medical device 800 may be a delivery device (e.g., the delivery device 202), and the rigidizable elements may rigidize a portion or the entire length of the delivery device. In some examples, the medical device 800 may be an instrument (e.g., the instrument 204), and the rigidizable elements may rigidize a portion (e.g., the rigidizable section 210) or the entire length of the instrument. The medical device 800 may include a flexible, tubular body 802 defining one or more tool or instrument channels 804. Various components may extend within the channel 804 including an imaging device 806 (e.g. an endoscopic camera), working conduit members 808, accessory channels 812, fluid channels 814, one or more selectively rigidizable elements 816, one or more selectively rigidizable elements 818, and/or one or more selectively rigidizable elements 820. In various examples, one or more of the components in the channel 804 may be omitted. For example a single rigidizable element may occupy an instrument channel with a single working conduit member. In other examples, additional rigidizable elements or components may extend with the lumen of the medical device. In examples where the medical device 800 is a delivery device, the working conduit members 808 may serve as working channels for instruments 204.

One or more of the selectively rigidizable elements may be transitioned to a rigid state to lock the current shape of the medical device 800. The shape-locked medical device may provide a stable platform for performing surgical, diagnostic, or therapeutic procedures. The selectively rigidizable element 816, for example, may include a layer around the working conduit 808. The layer may include, for example, a woven material (e.g., a mesh, braided, layered, or stent-like material) and a fluid conduit. In a flexible state, the woven material may bend and flex, allowing the underlying working conduit 808 to also bend and flex. The rigidizable element may be selectively rigidized and transitioned to a rigid state, for example, by a pneumatic actuator that applies a vacuum pressure to the fluid conduit to compress and rigidize the woven material around the working conduit 808. In other examples, a hydraulic actuator may deliver fluid to the fluid conduit to expand or inflate the fluid conduit, thereby rigidizing the woven material around the conduit 808. With the woven material of the rigidizable element 816 around the conduit 808 rigidized, the conduit 808 may become rigidized within the channel 804 and provide resistance to flexion or bending of the tubular body 802.

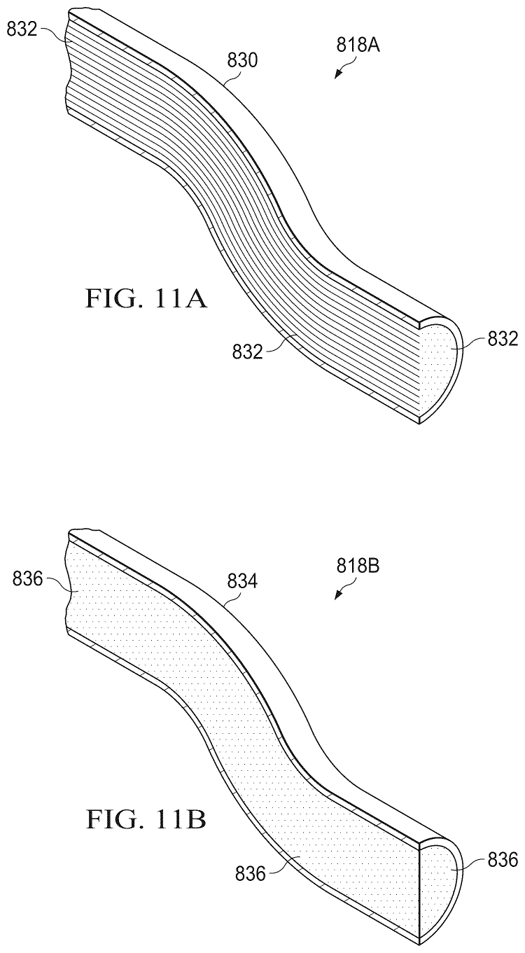

The selectively rigidizable element 818 may include a rigidizable rod. In some examples, the rigidizable rod 818 may include an inner flexible member and an outer rigidizable layer. The rigidizable layer may include, for example, a woven material (e.g., a mesh, braided, or stent-like material) and a fluid conduit. In a flexible state, the woven material may bend and flex, allowing the underlying inner flexible member to also bend and flex. The rigidizable element may be selectively rigidized and transitioned to a rigid state, for example, by a pneumatic or hydraulic actuator as described above. In some examples, the rigidizable rod may include a flexible sleeve filled with rigidizable material as shown in FIGS. 11A and 11B. In some embodiments, the rigidizable element 818 may be made of a plurality of rigidizable rods.

FIG. 11A illustrates a rigidizable element 818A which may be an example of the rigidizable element 818. The rigidizable element 818A may include a flexible outer housing or sleeve 830 filled with a plurality of flexible rods 832 (e.g., polymer, metal, or ceramic filaments). In a flexible state, the flexible sleeve 830 and the flexible rods 832 may bend and flex, allowing the entire rigidizable element 818A to also bend and flex with the tubular body 802. The rigidizable element 818A may be selectively rigidized and transitioned to a rigid state, for example, by a pneumatic actuator (e.g., a vacuum pressure) or a hydraulic actuator (e.g., fill with a fluid) that immobilizes the flexible rods 832 within the flexible sleeve 830. The rigidizable element 818A may be rigidized in a straight configuration or in a bent or curved configuration. In the rigidized state within the channel 804, the rigidizable element 818A may provide resistance to flexion or bending of the tubular body 802. In some examples, the flexible rods 832 may have further utility in addition to rigidization. For example, the flexible rods may be optical light guide fibers of an illumination system that convey light through the medical device 800.

FIG. 11B illustrates a rigidizable element 818B which may be an example of the rigidizable element 818. The rigidizable element 818B may include a flexible outer housing or sleeve 834 filled with a granular material 836 (e.g., polymer, metal, or ceramic beads). In a flexible state, the flexible sleeve 834 and may bend and flex and the granular material 836 may shift, allowing the entire rigidizable element 818B to also bend and flex with the tubular body 802. The rigidizable element 818B may be selectively rigidized and transitioned to a rigid state, for example, by a pneumatic actuator (e.g., a vacuum pressure) or a hydraulic actuator (e.g., fill with a fluid) that immobilizes the granular material 836 within the flexible sleeve 834. The rigidizable element 818B may be rigidized in a straight configuration or in a bent or curved configuration. In the rigidized state within the channel 804, the rigidizable element 818B may provide resistance to flexion or bending of the tubular body 802.

With reference to FIG. 10, the selectively rigidizable element 820 may be similar to any of the rigidizable elements 818, 818A, 818B. In this example, the rigidizable element 820 may have a variable, non-cylindrical shape sleeve that may conform to the surrounding components of the imaging system, working conduits, auxiliary channels, or other components within the channel 804 when rigidized to occupy the interstitial gaps between the other components within the channel 804. In some examples, the volume of the rigidizable element 820 may be larger than the volume of the rigidizable element 818, with the volume of the rigidizable element 820 allowed to conform to the spaces between the other components of the medical device. The rigidizable element 820 may be transitioned to a rigid state using any of the same pneumatic or hydraulic techniques previously described.

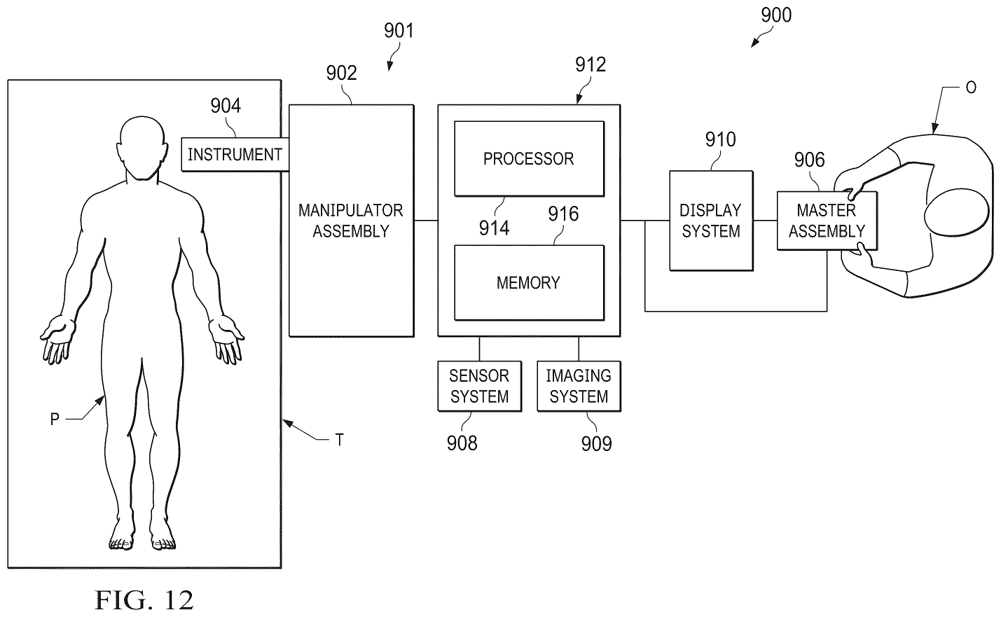

FIG. 12 illustrates a robotically-assisted medical system, according to some examples. As shown in FIG. 12, a robotically-assisted medical system 900 may include a manipulator assembly 902 for operating a medical instrument system 904 (e.g., medical instrument system 200 which may include the selective rigidization system 208) in performing various procedures on a patient P positioned on a table T in a surgical environment 901. The manipulator assembly 902 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 906, which may be inside or outside of the surgical environment 901, generally includes one or more control devices for controlling manipulator assembly 902. The control devices may include any number of a variety of input devices, such as joysticks, trackballs, data gloves, trigger-guns, hand-operated controllers, voice recognition devices, body motion or presence sensors, and/or the like. To provide the operator O a strong sense of directly controlling the medical instrument system 904, the control devices may be provided with the same degrees of freedom as the associated medical instrument system 904. In this manner, the control devices provide the operator O with telepresence or the perception that the control devices are integral with medical instrument system 904.

Manipulator assembly 902 supports medical instrument system 904 and may optionally include a plurality of actuators or motors that drive inputs on medical instrument system 904 in response to commands from a control system 912. The actuators may optionally include transmission or drive systems that when coupled to medical instrument system 904 may advance medical instrument system 904 into a naturally or surgically created anatomic orifice. Other transmission or drive systems may move the distal end of medical instrument system in multiple degrees of freedom, which may include three degrees of linear motion (e.g., linear motion along the X, Y, Z Cartesian axes) and in three degrees of rotational motion (e.g., rotation about the X, Y, Z Cartesian axes). For example the transmission systems may actuate control members of the instrument systems described herein. The manipulator assembly 902 may support various other systems for irrigation, treatment, or other purposes. Such systems may include fluid systems (including, for example, reservoirs, heating/cooling elements, pumps, and valves), generators, lasers, interrogators, and ablation components.

Robotically-assisted medical system 900 also includes a display system 910 for displaying an image or representation of the surgical site and medical instrument system 904 generated by an imaging system 909 which may include an endoscopic imaging system. Display system 910 and master assembly 906 may be oriented so an operator O can control medical instrument system 904 and master assembly 906 with the perception of telepresence. Any of the previously described graphical user interfaces may be displayable on a display system 910 and/or a display system of an independent planning workstation.

In some examples, the endoscopic imaging system components of the imaging system 909 may be integrally or removably coupled to medical instrument system 904. However, in some examples, a separate endoscope, attached to a separate manipulator assembly may be used with medical instrument system 904 to image the surgical site. The endoscopic imaging system 909 may be implemented as hardware, firmware, software, or a combination thereof which interact with or are otherwise executed by one or more computer processors, which may include the processors of the control system 912.

The sensor system 908 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 904. The sensor system 908 may also include temperature, pressure, force, or contact sensors or the like.

Robotically-assisted medical system 900 may also include control system 912. Control system 912 includes at least one memory 916 and at least one computer processor 914 for effecting control between medical instrument system 904, master assembly 906, sensor system 908, and display system 910. Control system 912 also includes programmed instructions (e.g., a non-transitory machine-readable medium storing the instructions) to implement instrument actuation using the robotically-assisted medical system including for navigation and steering.

Control system 912 may optionally further include a virtual visualization system to provide navigation assistance to operator O when controlling medical instrument system 904 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 912 may use a pre-operative image to locate the target tissue (using vision imaging techniques and/or by receiving user input) and create a pre-operative plan.

FIG. 13A is a simplified diagram of a medical instrument system 1000 according to some embodiments. In some embodiments, medical instrument system 1000 (e.g., medical instrument system 200) may be used as medical instrument system 904 in an image-guided medical procedure performed with teleoperated medical system 100. In some examples, medical instrument system 1000 may be used for non-teleoperational exploratory procedures or in procedures involving traditional manually operated medical instruments, such as endoscopy.

Medical instrument system 1000 includes elongate device 1002 (e.g. flexible delivery device 202), such as a flexible catheter, coupled to a drive unit 1004. Elongate device 1002 includes a flexible body 1016 having proximal end 1017 and distal end, or tip portion, 1018. In some embodiments, flexible body 1016 has an approximately 8-20 mm outer diameter. Other flexible body outer diameters may be larger or smaller.

Medical instrument system 1000 further includes a tracking system 1030 for determining the position, orientation, speed, velocity, pose, and/or shape of distal end 1018 and/or of one or more segments 1024 along flexible body 1016 using one or more sensors and/or imaging devices as described in further detail below. The entire length of flexible body 1016, between distal end 1018 and proximal end 1017, may be effectively divided into segments 1024. Tracking system 1030 may optionally be implemented as hardware, firmware, software or a combination thereof which interact with or are otherwise executed by one or more computer processors, which may include the processors of control system 912 in FIG. 12.

Tracking system 1030 may optionally track distal end 1018 and/or one or more of the segments 1024 using a shape sensor 1022. Shape sensor 1022 may optionally include an optical fiber aligned with flexible body 216 (e.g., provided within an interior channel (not shown) or mounted externally). In one embodiment, the optical fiber has a diameter of approximately 200 μm. In other embodiments, the dimensions may be larger or smaller. The optical fiber of shape sensor 1022 forms a fiber optic bend sensor for determining the shape of flexible body 1016. In one alternative, optical fibers including Fiber Bragg Gratings (FBGs) are used to provide strain measurements in structures in one or more dimensions. Sensors in some embodiments may employ other suitable strain sensing techniques, such as Rayleigh scattering, Raman scattering, Brillouin scattering, and Fluorescence scattering. In some embodiments, tracking system 230 may optionally and/or additionally track distal end 1018 using a position sensor system 1020, such as an electromagnetic (EM) sensor system. An EM sensor system may include one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 1020 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

Flexible body 1016 includes one or more channels 1021 sized and shaped to receive one or more medical instruments 1026 (e.g., instruments 204). In some embodiments, flexible body 1016 includes two channels 1021 for separate instruments 1026, however, a different number of channels 1021 may be provided. FIG. 13B is a simplified diagram of flexible body 1016 with medical instrument 1026 extended according to some embodiments. In some embodiments, medical instrument 1026 may be used for procedures such as surgery, biopsy, ablation, illumination, irrigation, or suction. Medical instrument 1026 can be deployed through channel 1021 of flexible body 1016 and used at a target location within the anatomy. Medical instrument 1026 may include, for example, image capture probes, biopsy instruments, laser ablation fibers, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. Medical instrument 1026 may be advanced from the opening of channel 1021 to perform the procedure and then retracted back into the channel when the procedure is complete. Medical instrument 1026 may be removed from proximal end 1017 of flexible body 1016 or from another optional instrument port (not shown) along flexible body 1016.

Medical instrument 1026 may additionally house cables, linkages, or other actuation controls (not shown) that extend between its proximal and distal ends to controllably the bend distal end of medical instrument 1026. Flexible body 1016 may also house cables, linkages, or other steering controls (not shown) that extend between drive unit 1004 and distal end 1018 to controllably bend distal end 1018 as shown, for example, by broken dashed line depictions 1019 of distal end 1018. In some examples, at least four cables are used to provide independent “up-down” steering to control a pitch of distal end 1018 and “left-right” steering to control a yaw of distal end 1018. In embodiments in which medical instrument system 1000 is actuated by a robot-assisted assembly, drive unit 1004 may include drive inputs that removably couple to and receive power from drive elements, such as actuators, of the teleoperational assembly. In some embodiments, medical instrument system 1000 may include gripping features, manual actuators, or other components for manually controlling the motion of medical instrument system 1000. The information from tracking system 1030 may be sent to a navigation system 1032 where it is combined with information from visualization system 1031 and/or the preoperatively obtained models to provide the physician or other operator with real-time position information.

In the description, specific details have been set forth describing some examples. Numerous specific details are set forth to provide a thorough understanding of the examples. It will be apparent, however, to one skilled in the art that some examples may be practiced without some or all these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions. Not all the illustrated processes may be performed in all examples of the disclosed methods. Additionally, one or more processes that are not expressly illustrated in may be included before, after, in between, or as part of the illustrated processes. In some examples, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging and treatment, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in examples of this disclosure may be implemented in software to execute on a processor of a computer system such as control processing system. When implemented in software, the elements of the examples of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative examples of the invention have been described and shown in the accompanying drawings, it is to be understood that such examples are merely illustrative of and not restrictive on the broad invention, and that the examples of the invention are not limited to the specific constructions and arrangements shown and described, since various other modifications may occur to those ordinarily skilled in the art.

1. A system comprising:
a flexible delivery device including a tool channel extending therethrough;
an elongated instrument configured to extend within the tool channel, the elongated instrument including a flexible section and a rigidizable section;
a selective rigidization system at least partially extending within the elongated instrument; and
a sensor system configured to determine position information for the rigidizable section relative to a distal portion of the delivery device,
wherein the selective rigidization system, responsive to the position information, is configured to transition a portion of the rigidizable section of the instrument from a bendable state to a rigid state.

2. The system of claim 1, wherein the flexible section remains in a flexible state when the rigidizable section is in the rigid state.

3. The system of claim 2, wherein when the rigidizable section is in the rigid state, the instrument is axially movable relative to the delivery device.

4. The system of claim 3, wherein when the rigidizable section is in the rigid state, the instrument is rotationally movable relative to the delivery device.

5. The system of claim 1, wherein the sensor system includes an optical fiber shape sensor.

6. The system of claim 1, wherein the sensor system includes an encoder.

7. The system of claim 1, wherein the sensor system includes an electromagnetic sensor.

8. The system of claim 1, wherein the sensor system includes an optical sensor.

9. The system of claim 1, wherein the portion of the rigidizable section includes an entire length of the instrument distal of the distal portion of the delivery device.

10. The system of claim 1, wherein the portion of the rigidizable section includes a partial length of the instrument distal of the distal portion of the delivery device.

11. The system of claim 1, wherein the portion of the rigidizable section includes a portion of the instrument distal to a distal end face of the delivery device and includes a portion of the instrument proximal to a distal end face of the delivery device.

12. The system of claim 1, wherein the rigidizable section includes predetermined segments and transitioning to the rigid state includes rigidizing one or more of the predetermined segments.

13.-14. (canceled)

15. The system of claim 1, wherein the selective rigidization system includes a tendon extending through a coil pipe in the rigidizable section.

16. The system of claim 1, wherein the selective rigidization system includes a series of compressible linkages.

17. The system of claim 1, wherein the selective rigidization system includes a pneumatic system configured to apply a vacuum to the rigidizable section.

18. The system of claim 1, wherein the selective rigidization system includes a hydraulic system configured to apply a pressurized fluid to the rigidizable section.

19. The system of claim 1, wherein the selective rigidization system includes a rigidizable element extending within the tool channel.

20. The system of claim 19, wherein the rigidizable element includes a stent or braided structure surrounding a working conduit or a flexible rod.

21. (canceled)

22. The system of claim 19, wherein the rigidizable element includes a flexible sleeve housing flexible rods, a granular material, or a plurality of flexible fiber optic light guides.

23.-24. (canceled)

25. The system of claim 1, wherein the selective rigidization system, responsive to the position information, is configured to transition a portion of the rigidizable section of the instrument from the rigid state to a bendable state.

26.-50. (canceled)
